(19) United States
(12) Patent Application Publication
(10) Pub. No.: US 2025/0163442 A1
(43) Pub. Date: May 22, 2025

(54) VISUAL PLASMID FOR REPLACING FLUORESCENT PROTEINS AND VISUALIZATION METHOD FOR EVALUATING PLANT PROTOPLASTS TRANSFORMATION AND TRANSFORMATION EFFICIENCY BY REPLACING FLUORESCENT PROTEINS

(71) Applicants: Institute of Cotton Research of the Chinese Academy of Agricultural Sciences, Anyang City (CN); National Nanfan Research Institute (Sanya), Chinese Academy of Agricultural Sciences, Sanya City (CN)

(72) Inventors: Guoli SONG, Anyang City (CN); Yunfei HAO, Anyang City (CN); Dongyun ZUO, Anyang City (CN); Hailiang CHENG, Anyang City (CN); Ji LIU, Anyang City (CN); Qiaolian WANG, Anyang City (CN); Yuanyuan LI, Anyang City (CN); Zhangcheng YUAN, Anyang City (CN)

(21) Appl. No.: 18/752,378

(22) Filed: Jun. 24, 2024

(30) Foreign Application Priority Data
Nov. 16, 2023 (CN) 202311522978X

(51) Int. Cl. C12N 15/82

(52) U.S. Cl. CPC C12N 15/8212

(57) ABSTRACT

A visual plasmid for replacing fluorescent proteins and visualization method for evaluating plant protoplasts transformation and transformation efficiency by replacing fluorescent proteins with visual plasmid. Genes expressed after the three kinds of plasmids are transformed into protoplasts metabolizes a substrate L-DOPA, causing protoplast cells to produce betacyanin or betaxanthin that are visible to the naked eye. The two pigments are visible to the naked eye under an optical microscope and may be used to intuitively determine whether a protoplast transformation experiment is successful. The plasmids can stably indicate successfully transformed cells. Their self-luminous intensities are superior to intensities of red fluorescent proteins tdtomato and EGFP, and can perfectly replace the role of the fluorescent proteins in evaluating protoplast transformation efficiency. This technology greatly simplifies the protoplast transformation experiment, gets rid of dependence on a fluorescent light source, and lowers the threshold for using the protoplast transformation technology.

### REFERENCE TO AN ELECTRONIC SEQUENCE LISTING

The content of the electronic sequence listing (SEQUENCE-0213-0317PUS1.xml; Size: 13,388 bytes; and Date of Creation: Jun. 24, 2024) is herein incorporated by reference in its entirety.

### TECHNICAL FIELD

This application belongs to the technical field of bioengineering, and particularly relates to three kinds of visual plasmids for replacing fluorescent proteins and preparation methods therefor and applications thereof, as well as a visualization method for evaluating plant protoplasts transformation and transformation efficiency by replacing fluorescent proteins with visual plasmids.

### BACKGROUND

Plant protoplasts may be used for transient genetic transformation, somatic cell fusion and plant regeneration in the field of bioengineering. At present, a PEG-mediated transient transformation technology of protoplasts has been successfully established in many species. This technology may be applied to positioning and interaction of biological macromolecules, development of gene editing tools, and single-cell sequencing, etc. The transient transformation technology of protoplasts includes four main steps: isolation, purification and transformation of protoplasts, and transformation efficiency evaluation. The transformation efficiency evaluation is an essential quality control step in a protoplast transformation experiment. It is understood that almost all current evaluation methods are based on fluorescent proteins, such as GFP, RFP, mCherry, etc. The main principle is that successfully transformed protoplast cells express the fluorescent proteins on the plasmids and produce fluorescence under fluorescent excitation light. The transformation efficiency can be calculated by comparing with non-fluorescent cells. However, in actual operations, it is often only necessary to know whether the transformation is successful to eliminate problems with reagent preparation and operation errors, so as to terminate subsequent experiments in time to avoid wasting time. In this fluorescent protein-based method, observation cannot be performed quickly under an ordinary optical microscope and this method relies on expensive excitation light source equipment and additional operating steps. At present, there is no mature application technology that uses non-destructive and naked eye-visible technical methods to evaluate the transformation efficiency of the protoplasts.

Betacyanin and betaxanthin are plant pigments unique to plants of Caryophyllales. Their synthesis pathway uses tyrosine as a substrate and is hydroxylated by P450 oxidase (CYP76AD1) into L-3,4-dihydroxyphenylalanine (L-DOPA). The L-DOPA may be further oxidized to cyclo-DOPA by CYP76AD1. The L-DOPA may also be transformed into betalamic acid by L-DOPA 4,5-dioxygenase (DODA). On the one hand, the betalamic acid may combine with cyclo-DOPA to produce betacyanin under the catalysis of glucosyltransferase (GT). On the other hand, it can react spontaneously with amino acids or amino groups to form betaxanthin. Therefore, theoretically, only three genes: CYP76AD1, DODA, and GT may be needed to reconstruct the metabolic pathways of betacyanin and betaxanthin in vivo. In recent years, relevant research has used a betalain system to fuse and express the three genes through self-cleaving polypeptides to form a synthetic Ruby gene, which has been successfully applied to visual screening of transgenic plants. At present, this denovo synthesis strategy has been successfully applied not only in plants such as tobacco and cotton, but also in yeast and silkworms. In addition, betacyanin and betaxanthin not only have red and yellow colors visible to the naked eye, but also have corresponding strong autofluorescence within the excitation wavelength range of green fluorescent proteins and red fluorescent proteins. However, the Ruby gene has not been used as a reporter gene in protoplasts in existing studies. There may be the following four main reasons: (1) the Ruby gene is up to 3951 bp long, and in most protoplast transformation systems, the larger the plasmid is, the lower the transformation efficiency is; (2) studies have shown that the expression level of genes located behind the self-cleaving polypeptide will be lower than the genes in front, and accordingly, expressing Ruby through a single expression cassette may not accumulate enough betacyanin in such a transient protoplast transformation system; (3) differing from transgenic plants where a Ruby metabolic precursor substance tyrosine is continuously present, there is no additional source of tyrosine after protoplast isolation, and meanwhile, a variety of intermediate metabolites are involved in the expression process of the Ruby gene; there may be intermediate metabolites that are transported out of the cells or enter other metabolic directions, such that sufficient betalain pigments cannot be accumulated in the end, resulting in no visible cell discoloration; and (4) although supplementation may be achieved by exogenously applying substrates at this stage, no or very few cells transformed with Ruby appear to show color using conventional application methods (such as application during an incubation phase after transformation). In summary, existing technologies cannot stably accumulate and use betacyanin in protoplasts through plasmid transformation.

### SUMMARY

In view of this, the present disclosure provides three kinds of visual plasmids for replacing fluorescent proteins and preparation methods therefor and applications thereof, as well as a visualization method for evaluating plant protoplasts transformation and transformation efficiency by replacing fluorescent proteins with visual plasmids. Compared with the limitations of current fluorescent protein reporting systems in their dependence on excitation light source equipment, the plasmids, the visualization method and related applications provided by the present disclosure are a non-destructive, low-cost, and naked eye-visible protoplast transformation reporting system, which produces betacyanin and betaxanthin using a metabolic substrate L-DOPA, and a novel protoplast transformation reporting system is designed. The system is named a BTX reporting system in the present disclosure.

In order to achieve the above objects, the present disclosure adopts the following technical solution:
the present disclosure provides visual plasmids for replacing fluorescent proteins, and the visual plasmids include pUC-Ruby, pUC-tdDODA, and pUC-DODA;
genes for replacing the fluorescent protein in the visual plasmid pUC-Ruby include a Ruby gene;
in the present disclosure, the Ruby gene includes a CYP76AD1 gene, a DODA gene, and a GT gene;
the amino acid sequence of the CYP76AD1 protein expressed by the CYP76AD1 gene is shown in SEQ ID NO.1 in a patent CN 112063649 A;
the DODA gene is shown in SEQ ID NO.2;
the amino acid sequence of the GT protein expressed by the GT gene is shown in SEQ ID NO. 3 in a patent CN 112063649 A;
genes for replacing the fluorescent protein in the visual plasmid pUC-tdDODA include, for example, a tdTomato gene shown in SEQ ID NO. 1 and a DODA gene shown in SEQ ID NO.2; and
genes for replacing the fluorescent protein in the visual plasmid pUC-tdDODA include, for example, a DODA gene shown in SEQ ID NO.2.

The genes expressed after the three kinds of plasmids provided by the present disclosure are transformed and entered into protoplasts metabolizes the substrate L-DOPA, causing the protoplast cells to produce betacyanin and betaxanthin that are visible to the naked eye. These two pigments are visible to the naked eye under an optical microscope and may be used to intuitively determine whether a protoplast transformation experiment is successful.

In the present disclosure, a vector skeleton of the visual plasmid includes a vector skeleton of pUC series; and the vector skeleton further includes a promoter 35S and a terminator NOS. The vector skeleton preferably used in the present disclosure is pUC-2×35S-EGFP.

In the present disclosure, the tdTomato gene and the DODA gene in the visual plasmid pUC-tdDODA are connected by a linkage unit shown in SEQ ID NO 3. In the present disclosure, the tdTomato gene and the DODA gene are connected in any order; the DNA linkage unit is a self-cleaving polypeptide sequence; further preferably, the DNA linkage unit is a self-cleaving polypeptide F2A sequence shown in SEQ ID NO.3.

In the present disclosure, primers for amplifying the RUBY gene are shown in SEQ ID NO.4 and SEQ ID NO 5;
primers for amplifying the tdTomato gene are shown in SEQ ID NO.6 and SEQ ID NO.7;
primers for amplifying the DODA gene with a DNA linkage unit are shown in SEQ ID NO.8 and SEQ ID NO.9;
primers for amplifying the DODA gene are shown in SEQ ID NO.10 and SEQ ID NO.11.

The present disclosure provides an application of any of the above visual plasmids for replacing fluorescent proteins in determining whether plant protoplast transformation is successful.

The present disclosure provides an application of any of the above visual plasmids for replacing fluorescent proteins in evaluating a plant protoplast transformation efficiency.

The present disclosure provides a visualization method for evaluating plant protoplasts transformation and transformation efficiency by replacing fluorescent proteins. The method includes preparation and transformation processes of a protoplast transformation solution;
the protoplast transformation solution includes L-DOPA at a concentration of 10 mM; and
the transformation process is as follows: the visual plasmid and substrate of any one of above-described are co-transformed and entered into cells.

In the present disclosure, a system of the protoplast transformation solution is shown in Table 1.

The present disclosure provides supporting protoplast transformation reagents and transformation methods. When the above three kinds of plasmids are transformed without adding the substrate L-DOPA, the betalaine reporting system cannot work normally. However, when the L-DOPA is added during an overnight incubation stage, the cells cannot uptake the substrate efficiently and therefore cannot work stably. To this end, the present disclosure adopts the method of co-transformation of the substrate and plasmid into the protoplasts. After the transformation, the untaken substrate is replaced by a W5 resuspension. The W5 solution system is shown in Table 2.

TABLE 1
System of protoplast transformation solution

| Component | Concentration | |
|---|---|---|
| Mannitol | 200 | mM |
| CaCl₂ | 100 | mM |
| PEG4000 | 40% | |
| Substrate L-DOPA | 10 | mM |
| Supplementing double | 5 | mL |

distilled water to

TABLE 2
W5 solution system

| Component | Concentration | |
|---|---|---|
| MES | 20 | mM |
| CaCl₂ | 125 | mM |
| NaCl | 154 | mM |
| KCl | 5 | mM |
| Supplementing double | 100 | mL |

distilled water to

The present disclosure also provides an application of the above visualization method in improving the efficiency of substrate uptake by protoplasts.

The present disclosure also provides an application of the above visualization method in determining whether protoplast transformation is successful.

The present disclosure also provides an application of the above visualization method in evaluating protoplast transformation efficiency.

The actual using effects of the reporting system provided by the present disclosure and commonly used fluorescent proteins in judging the protoplast transformation efficiency can be known from the examples. The two plasmids pUC-tdDODA and pUC-DODA provided by the present disclosure are used. The two plasmids may be used to compare the betalaine color and the intensity and stability of betalaine's autoluminescence with that of fluorescent proteins.

The function of the pUC-tdDODA plasmid is that by placing tdTomato and DODA under the same expression cassette and transcribing and translating them simultaneously, the brightness and stability of the two reporting systems, betalaine and fluorescent proteins, may be more accurately compared. The brightness and photostability of the red fluorescent protein tdTomato are significantly superior to those of other commonly used fluorescent proteins such as EGFP, mCherry, and Venus.

The function of the pUC-DODA plasmid is to, compared with pUC-tdDODA, further increase the expression level of the DODA gene. Because some studies have shown that the protein expression behind the self-cleaved polypeptide will be slightly reduced. By transforming pUC-DODA and pUC-EGFP respectively, the transformation efficiencies calculated by the two reporting systems DODA and EGFP can be compared.

The beneficial effects of the present disclosure are as follows:
the present disclosure utilizes betalaine metabolites to evaluate protoplast transformation and transformation efficiency. Each of the reporting systems pUC-tdDODA and pUC-DODA based on betalaine in the present disclosure can stably indicate successfully transformed cells, and their self-luminous intensity is superior to that of red fluorescent proteins tdtomato and EGFP, and can perfectly replace fluorescent proteins in evaluating the protoplasts transformation efficiency. The emergence of this technology greatly simplifies protoplast transformation experiments, gets rid of the dependence on fluorescent light sources, and lowers the threshold for using protoplast transformation technology. The metabolic flow chart of betalaine and betaxanthin is shown in FIG. 1.

### BRIEF DESCRIPTION OF THE DRAWINGS

FIG. 1 is a metabolic flow chart of betalaine accumulation in protoplasts;

FIG. 2 is a diagram of accumulated betalaine after protoplasts are transformed with a pUC-Ruby plasmid; the left diagram shows red cells where betalaine is mainly accumulated, and the right diagram shows yellow cells where betaxanthin is accumulated;

FIG. 3 is a schematic diagram of a pUC-tdDODA plasmid;

FIG. 4 shows results after overnight incubation of the protoplasts transformed with a tdDODA plasmid and a substrate;

FIG. 5 shows results of overnight incubation of the protoplasts transformed with the tdDODA plasmid without adding the substrate;

FIG. 6 shows results of overnight incubation of the protoplasts transformed with the tdDODA plasmid adding the substrate;

FIG. 7 is a schematic diagram of the pUC-DODA plasmid; and

FIG. 8 shows that, after protoplasts are transformed with the pUC-DODA plasmid, the successfully transformed cells turn yellow and autofluorescence of betalaine appears under excitation light.

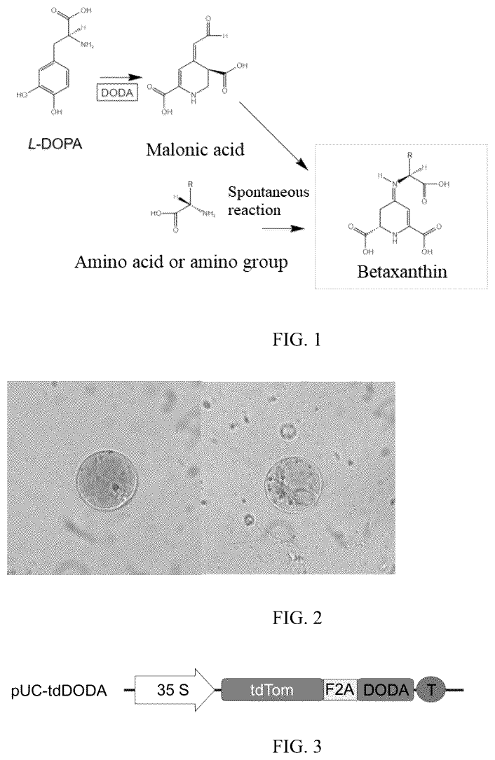

### DETAILED DESCRIPTION

The present application will be further explained below in conjunction with examples.

Before introducing specific examples, some experimental background conditions in the following examples are briefly introduced as follows.

Biomaterials:
a vector pUC-2×35S-EGFP stored in a laboratory;
an Escherichia coli strain DH5a Chemically Competent Cell purchased from Shanghai Vidy Biotechnology Co., Ltd.;
relevant sequence synthesis and sequencing work provided and completed by Genscript Biotechnology Co., Ltd. and Sangon Biotech (Shanghai) Co., Ltd.

Main reagents:
an agarose gel DNA recovery kit (enhanced type) and an ultra-thin DNA product purification kit for recovering PCR products and vectors, purchased from Beijing Tiangen Biotechnology Company;
the kit for the recombination reaction, ClonExpress® II One Step Cloning Kit, purchased from Nanjing Vazyme Company;
relevant restriction enzymes, all the products of New England Biolabs (NEB);
an endotoxin-free plasmid maximal extraction kit for performing a plasmid extraction, purchased from Beijing Tiangen Biotechnology Company; and
L-DOPA purchased from Sangon Biotech (Shanghai) Co., Ltd.

### Example 1

Whether protoplast transformation is successful is determined by using a reporting system based on the betalaine.

In this example, by constructing a Ruby gene into pUC-2×35S-EGFP, and metabolizing exogenously applied L-DOPA, betalaine visible to the naked eye is produced, which is used to determine whether the protoplast transformation is successful.

A specific implementation process is briefly introduced as follows.

### (1) Reporting Plasmid Construction

A PCR amplification was performed with the following primer pairs by means of using a RUBY gene as a DNA template:

PUC-RUBY F:
SEQ ID NO. 4
5′-TTTACGAACGATAGACTAGTATGGATCATGCTACTCTTGCTATGAT
T TTG-3′,;
PUC-RUBY R:
SEQ ID NO. 5
5′-GATCGGGGAAATTCTCTAGATCATTGCAAAGAAGGCTCAAGTTTA
GC-3′,.

TABLE 3
PCR reaction system

| Component | Volume | |
|---|---|---|
| Phanta Max Super-Fidelity DNA Polymerase | 1 | μL |
| 2 × Phanta Max Buffer | 10 | μL |
| dNTP Mix | 0.5 | μL |
| Upstream primer RUBY F | 0.8 | μL |
| Downstream primer RUBY R | 0.8 | μL |
| Template DNA | 1 | μL |
| Supplementing double distilled water to | 20 | μL |

TABLE 4
PCR amplification program

| Step | Temperature | Time | | Cycle number |
|---|---|---|---|---|
| Predenaturation | 95° C. | 5 | min | 1 |
| Denaturation | 95° C. | 15 | sec | 33 |
| Annealing | 58° C. | 15 | sec | |
| Extension | 72° C. | 45 | sec | |
| Extension | 72° C. | 5 | min | 1 |

An amplification was performed to obtain a PCR product with a length of 3991 bp and the product was recovered by using an agarose gel DNA recovery kit (enhanced type).

The skeleton plasmid of the vector was pUC-2×35S-EGFP. Restriction enzymes SpeI and XbaI were used to linearize the vector and cut out an EGFP gene.

TABLE 5
Enzyme cutting system is as follows

| Component | Volume | |
|---|---|---|
| 10x Cut smart buffer | 10 | μL |
| SpeI | 1 | μL |
| XbaI | 1 | μL |
| Vector | 2000 | ng |
| Supplementing double | 100 | μL |
| distilled water to | | |

A reaction system was placed in a 37° C. incubator for enzyme cutting overnight.

Further, the linearized vector was recovered by using an ultra-thin DNA product purification kit.

Further, the PCR product and the linearized vector were recombined by using a ClonExpress® II One Step Cloning Kit.

Further, the recombinant product was transformed and entered into E. coli strain DH5a. After overnight growth on a plate, monoclones were selected for colony PCR identification and positive transformants are screened.

Further, whether the RUBY gene was correctly constructed into the pUC-2×35S-EGFP plasmid was confirmed by using a Sanger sequencing method.

### (2) Protoplast Transformation

The pUC-RUBY plasmid was extracted by using an endotoxin-free plasmid maxiprep kit.

10 μg of the pUC-Ruby plasmid was added to 100 μL of extracted and isolated cotton protoplasts (about 10,000 cells), and the mixture was flicked, uniformly mixed, and placed in the dark for 10 minutes. Then a protoplast transformation solution was added, the mixture was flicked, uniformly mixed, and placed in the dark for 30 minutes. The W5 solution was added to terminate the reaction, the mixture was centrifugalized to discard the transformation solution, and the transformed protoplasts were resuspended with the W5 solution. The W5 solution system is shown in Table 2 above.

### (3) Observation of Transformation Results

After the transformed protoplasts were incubated overnight at room temperature, cells were observed under an optical microscope equipped with a fluorescence system. As shown in FIG. 2, light red, red and yellow cells appear in the transformed protoplasts to indicate that the protoplast cells express the ingested pUC-RUBY plasmid and successfully accumulate different concentrations of betacyanin and betaxanthin. This marks the success of this protoplast transformation experiment.

### Example 2

The actual effects of a betaxanthin system and a red fluorescent protein system in counting transformation efficiency are compared.

In this example, the pUC-tdDODA plasmid was used to express a tdDODA fusion gene. The tdDODA fusion gene directly metabolized exogenously applied L-DOPA to produce betaxanthin visible to the naked eye, and at the same time, produced almost the same amount of red fluorescent protein tdTomato. By counting the ratio of yellow and red light cells under visible light, as well as the ratio of betaxanthin's spontaneous green and red light cells, the actual effects of the betaxanthin system and the red fluorescent protein system in counting transformation efficiency can be compared. The schematic diagram of the pUC-tdDODA plasmid is shown in FIG. 3.

A specific implementation process is briefly introduced as follows.

### (1) Reporting Plasmid Construction

By using the PLV23 (TDTOMATO) plasmid as a DNA template to amplify TDTOMATO, the following primer pairs were used in the PCR reaction:

PUC-TD F:
SEQ ID NO. 6
5′-TTTACGAACGATAGACTAGTATGGTGAGCAAGGGCGAGG-3′,;
MID-TD R:
SEQ ID NO. 7
5′-CTTATACAGCTCGTCCATGCCGT-3′,.

By using the RUBY plasmid as a DNA template to amplify F2A and DODA, the following primer pairs were used in the PCR reaction:

MID-DODA F:
SEQ ID NO. 8
5′-GCATGGACGAGCTGTATAAGGGTTTCAGGAGCTACAAATTTTTCTC
TT-3′,;
PUC-DODA R:
SEQ ID NO. 9
5′-CGATCGGGGAAATTCTCTAGATTAAGCAGATGTGAACTTGTATGA
TC-3′,.

TABLE 6
PCR reaction system

| Component | Volume | |
|---|---|---|
| Phanta Max Super-Fidelity DNA Polymerase | 1 | μL |
| 2 × Phanta Max Buffer | 10 | μL |
| dNTP Mix | 0.5 | μL |
| Upstream primer | 0.8 | μL |
| Downstream primer | 0.8 | μL |
| Template plasmid | 1 | μL |
| Supplementing double distilled water to | 20 | μL |

TABLE 7
PCR amplification program

| Step | Temperature | Time | | Cycle number |
|---|---|---|---|---|
| Predenaturation | 95° C. | 5 | min | 1 |
| Denaturation | 95° C. | 15 | sec | 33 |
| Annealing | 58° C. | 15 | sec | |
| Extension | 72° C. | 45 | sec | |
| Extension | 72° C. | 5 | min | 1 |

The two amplified PCR products were recovered by using the agarose gel DNA recovery kit (enhanced type).

The skeleton plasmid of the vector is pUC-2×35S-EGFP. Restriction enzymes SpeI and XbaI were used to linearize the vector and cut out the EGFP gene.

TABLE 8
Enzyme cutting system is as follows

| Component | Volume | |
|---|---|---|
| 10x Cut smart buffer | 10 | μL |
| SpeI | 1 | μL |
| XbaI | 1 | μL |
| Vector | 2000 | ng |
| Supplementing double | 100 | μL |

distilled water to

The reaction system was placed in a 37° C. incubator for enzyme cutting overnight.

Further, the linearized vector was recovered by using an ultra-thin DNA product purification kit.

Further, the two PCR products and the linearized vector were recombined by using a ClonExpress® II One Step Cloning Kit.

Further, the recombinant product was transformed and entered into E. coli strain DH5a. After overnight growth on a plate, monoclones were selected for colony PCR identification and positive transformants are screened.

Further, whether the tdDODA gene was correctly constructed into the pUC-2×35S plasmid was confirmed by using a Sanger sequencing method.

### (2) Protoplast Transformation

The pUC-tdDODA plasmid was extracted by using an endotoxin-free plasmid maxiprep kit.

10 μg of pUC-tdDODA plasmid was added to 100 μL of extracted and isolated cotton protoplasts (about 10,000 cells), and the mixture was flicked, uniformly mixed, and placed in the dark for 10 minutes. Then a protoplast transformation solution was added, and the mixture was flicked, uniformly mixed, and placed in the dark for 30 minutes. A W5 solution was added to terminate the reaction, the mixture was centrifugalized to discard the transformation solution, and the transformed protoplasts were resuspended in the W5 solution.

### (3) Observation of Transformation Results

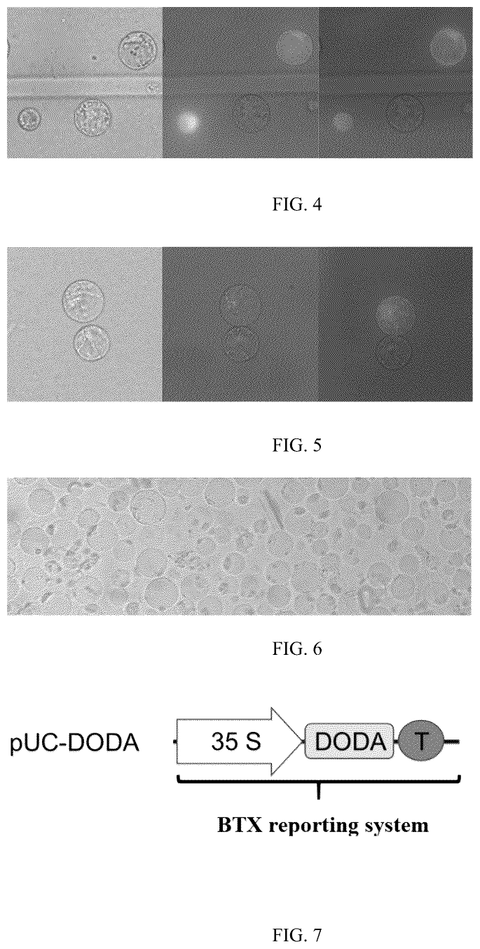

After the transformed protoplasts were incubated overnight at room temperature, cells were observed under an optical microscope equipped with a fluorescence system. After adding the substrate L-DOPA during the transformation process, cells successfully transformed with pUC-tdDODA show red fluorescence under 540-580 nm excitation light and bright betaxanthin autoluminescence under 465-495 nm excitation light. In bright field, a yellowish color visible to the naked eye appears below (refer to FIG. 4 here). However, cells that are successfully transformed with the pUC-tdDODA without adding the substrate L-DOPA only show the red fluorescence of tdTomato (refer to FIG. 5 here). No yellow cells have been seen after adding the substrate L-DOPA during the incubation phase (refer to FIG. 6 here).

As shown statistically in Table 9, more than 50% of the cells with red fluorescence appear yellow visible to the naked eye, and almost 100% show bright betaxanthin autoluminescence. This shows that the betaxanthin reporting system has similar stability and expression intensity to tdTomato red fluorescence, and the transformation method of the present disclosure successfully enables cells to efficiently uptake the substrate L-DOPA.

TABLE 9
Relative cell number (%)

| | Bright field | | |
|---|---|---|---|
| | (yellow) | 465-495 nm | 540-580 nm |
| Mean ± SD | 54.73 ± 14.88 | 95.23 ± 8.26 | 100 |

### Example 3

The actual effects of a betaxanthin system and a green fluorescent protein system in counting protoplast transformation efficiency are compared.

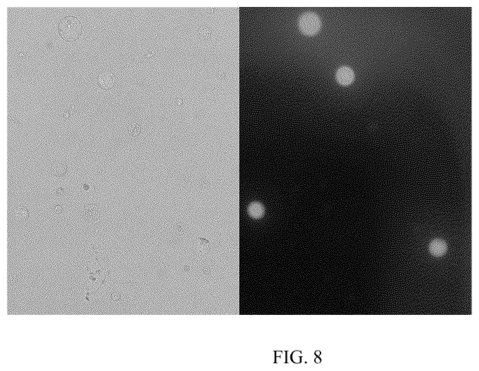

In this example, the pUC-DODA and pUC-EGFP plasmids are used to conduct protoplast transformation experiments. After the protoplast expresses the DODA gene, it directly metabolizes the exogenously applied L-DOPA to produce betaxanthin visible to the naked eye. By comparing the naked eye color and fluorescent cell ratio of the protoplasts transformed with the pUC-EGFP, the actual effects of the betaxanthin system and the yellow fluorescent protein system in counting protoplast transformation efficiency can be compared. The schematic diagram of the pUC-DODA plasmid is shown in FIG. 7.

A specific implementation process is briefly introduced as follows.

### (1) Reporting Plasmid Construction

By using a Ruby plasmid as a DNA template to amplify DODA, the following primer pairs were used in the PCR reaction:

PUC-DODA F:
SEQ ID NO. 10
5′-TTTACGAACGATAGACTAGTATGAAGATGATGAATGGTGAAGAT-
3′,;
PUC-DODA R:
SEQ ID NO. 11
5′-CGATCGGGGAAATTCTCTAGATTAAGCAGATGTGAACTTGTATGAT
C-3′,.

TABLE 10
PCR reaction system

| Component | Volume | |
|---|---|---|
| Phanta Max Super-Fidelity DNA Polymerase | 1 | μL |
| 2 × Phanta Max Buffer | 10 | μL |
| dNTP Mix | 0.5 | μL |
| Upstream primer | 0.8 | μL |
| Downstream primer | 0.8 | μL |
| Template DNA | 1 | μL |
| Supplementing double distilled water to | 20 | μL |

TABLE 11
PCR amplification program

| Step | Temperature | Time | | Cycle number |
|---|---|---|---|---|
| Predenaturation | 95° C. | 5 | min | 1 |
| Denaturation | 95° C. | 15 | sec | 33 |
| Annealing | 58° C. | 15 | sec | |
| Extension | 72° C. | 30 | sec | |
| Extension | 72° C. | 5 | min | 1 |

The two amplified PCR products were recovered by using an agarose gel DNA recovery kit (enhanced type).

The skeleton plasmid of the vector is pUC-2×35S-EGFP. Restriction enzymes SpeI and XbaI were used to linearize the vector and cut out the EGFP gene.

TABLE 12
Enzyme digestion system

| Component | Volume | |
|---|---|---|
| 10x Cut smart buffer | 10 | μL |
| SpeI | 1 | μL |
| XbaI | 1 | μL |
| Vector | 2000 | ng |
| Supplementing double | 100 | μL |
| distilled water to | | |

The reaction system was placed in a 37° C. incubator for enzyme cutting overnight.

Further, the linearized vector was recovered by using an ultra-thin DNA product purification kit.

Further, the PCR product and linearized vector were recombined by using a ClonExpress® II One Step Cloning Kit.

Further, the recombinant product was transformed and entered into E. coli strain DH5a. After overnight growth on a plate, monoclones were selected for colony PCR identification and positive transformants are screened.

Further, whether the DODA gene was correctly constructed into the pUC-2×35S-EGFP plasmid was confirmed by using a Sanger sequencing method.

### (2) Protoplast Transformation

The pUC-DODA plasmid was extracted by using an endotoxin-free plasmid maxiprep kit.

10 μg of pUC-DODA and pUC-2×35S-EGFP plasmids were respectively added to 100 μL of extracted and isolated cotton protoplasts (approximately 10,000 cells), and the mixture was flicked, uniformly mixed, and placed in the dark for 10 min. Then a protoplast transformation solution was added, and the mixture was flicked, uniformly mixed, and placed in the dark for 30 min. A W5 solution was added to terminate the reaction, the mixture was centrifugalized to discard the transformation solution, and the transformed protoplasts were resuspended in the W5 solution.

### (3) Observation of Transformation Results

After the transformed protoplasts were incubated overnight at room temperature, cells were observed under an optical microscope equipped with a fluorescence system. As shown in FIG. 8, yellow cells appear in the transformed protoplasts under the bright field view. When the fluorescence excitation light source is switched to 540-580, the yellow cells also have bright autofluorescence. The transformation efficiency under different conditions and time periods is shown in Table 13.

TABLE 13
Transformation efficiency under different conditions and time periods

| | Bright field | 465-495 | Bright field | 465-495 | 465-495 |
|---|---|---|---|---|---|
| | (yellow cells) | nm | (yellow cells) | nm | nm |
| | illumination | illumination | darkness | darkness | EGFP |
| 12 h | 14.40 ± 1.90 | 18.87 ± 5.10 | 26.63 ± 3.35 | 42.20 ± 1.91 | 44.40 ± 1.90 |
| 18 h | 14.43 ± 5.09 | 19.97 ± 3.35 | 35.50 ± 1.90 | 41.07 ± 3.87 | 34.43 ± 5.10 |
| 24 h | 24.40 ± 6.96 | 31.07 ± 6.93 | 33.30 ± 3.30 | 36.67 ± 5.77 | 29.97 ± 3.35 |

The statistical transformation efficiencies of pUC-DODA and pUC-2×35S-EGFP are compared at different time points after transformation and under illumination and darkness.

The results show that the transformation efficiency of cells transformed with pUC-DODA under darkness incubation is higher than that counted under illumination, and as time increases, the number of yellow cells gradually increases under the bright field. At 12 hours after transformation, the transformation efficiency under the bright field is about half of that under fluorescence, but there is no significant difference between the two at 18 hours. At three time points, the counted transformation efficiency of pUC-DODA under fluorescence under darknessis similar or even better than that of EGFP. The results show that the pUC-DODA plasmid can perfectly replace the pUC-2×35S-EGFP plasmid to evaluate the protoplast transformation efficiency, and the betaxanthin-based pUC-DODA system can complete the counting only under an optical microscope, which gets rid of the limitation to fluorescent light sources.

The above is only preferred examples of the present invention. It should be noted that those skilled in the art can further make several improvements and modifications without departing from the principle of the present invention. The improvements and modifications should also be regarded within the scope of protection of the present invention.

Sequence table

| | |
|---|---|
| <110> | Sanya National Institute of Southern Transplantation, Chinese Academy of |
| | Agricultural Sciences |
| | Cotton Research Institute, Chinese Academy of Agricultural Sciences |
| <120> | Visual plasmid for replacing fluorescent proteins and visualization method for |
| | evaluating plant protoplasts transformation and transformation efficiency by |
| | replacing the fluorescent proteins |
| <141> | 2023-11-07 |
| <160> | 11 |
| <170> | SIPOSequenceListing 1. 0 |
| <210> | 1 |
| <211> | 1428 |
| <212> | DNA |
| <213> | Artificial Sequence |
| <400> | 1 |

| | |
|---|---|
| atggtgagca agggcgagga ggtcatcaaa gagttcatgc gcttcaaggt gcgcatggag | 60 |
| ggctccatga acggccacga gttcgagatc gagggcgagg gcgagggccg cccctacgag | 120 |
| ggcacccaga ccgccaagct gaaggtgacc aagggcggcc ccctgccctt cgcctgggac | 180 |
| atcctgtccc cccagttcat gtacggctcc aaggcgtacg tgaagcaccc cgccgacatc | 240 |
| cccgattaca agaagctgtc cttccccgag ggcttcaagt gggagcgcgt gatgaacttc | 300 |
| gaggacggcg gtctggtgac cgtgacccag gactcctccc tgcaggacgg cacgctgatc | 360 |
| tacaaggtga agatgcgcgg caccaacttc ccccccgacg gccccgtaat gcagaagaag | 420 |
| accatgggct gggaggcctc caccgagcgc ctgtaccccc gcgacggcgt gctgaagggc | 480 |
| gagatccacc aggccctgaa gctgaaggac ggcggccact acctggtgga gttcaagacc | 540 |
| atctacatgg ccaagaagcc cgtgcaactg cccggctact actacgtgga caccaagctg | 600 |
| gacatcacct cccacaacga ggactacacc atcgtggaac agtacgagcg ctccgagggc | 660 |
| cgccaccacc tgttcctggg gcatggcacc ggcagcaccg gcagcggcag ctccggcacc | 720 |
| gcctcctccg aggacaacaa catggccgtc atcaaagagt tcatgcgctt caaggtgcgc | 780 |
| atggagggct ccatgaacgg ccacgagttc gagatcgagg gcgagggcga gggccgcccc | 840 |
| tacgagggca cccagaccgc caagctgaag gtgaccaagg gcggccccct gcccttcgcc | 900 |
| tgggacatcc tgtcccccca gttcatgtac ggctccaagg cgtacgtgaa gcaccccgcc | 960 |
| gacatccccg attacaagaa gctgtccttc cccgagggct tcaagtggga gcgcgtgatg | 1020 |
| aacttcgagg acggcggtct ggtgaccgtg acccaggact cctccctgca ggacggcacg | 1080 |
| ctgatctaca aggtgaagat gcgcggcacc aacttccccc ccgacggccc cgtaatgcag | 1140 |
| aagaagacca tgggctggga ggcctccacc gagcgcctgt acccccgcga cggcgtgctg | 1200 |
| aagggcgaga tccaccaggc cctgaagctg aaggacggcg gccactacct ggtggagttc | 1260 |
| aagaccatct acatggccaa gaagcccgtg caactgcccg gctactacta cgtggacacc | 1320 |
| aagctggaca tcacctccca caacgaggac tacaccatcg tggaacagta cgagcgctcc | 1380 |
| gagggccgcc accacctgtt cctgtacggc atggacgagc tgtataag | 1428 |

| | |
|---|---|
| <210> | 2 |
| <211> | 825 |
| <212> | DNA |
| <213> | Artificial Sequence |
| <400> | 2 |

| | |
|---|---|
| atgaagatga tgaatggtga agatgctaac gatcaaatga tcaaggagtc tttctttatc | 60 |
| acccatggaa accctatcct tactgttgaa gatacacatc cattgaggcc tttctttgaa | 120 |
| acttggagag agaaaatctt ctcaaagaaa cctaaggcta ttcttattat ctctggtcat | 180 |
| tgggagaccg ttaagccaac tgttaatgct gttcatatca acgatactat ccatgatttc | 240 |
| gatgattacc ctgctgctat gtatcaattc aagtacccag ctcctggaga accagagctt | 300 |
| gctaggaagg ttgaagagat cttgaagaaa tcaggttttg aaacagctga gaccgatcaa | 360 |
| aagagaggtt tggatcatgg agcttgggtt cctttgatgc ttatgtaccc agaagctgat | 420 |
| attcctgttt gtcaactttc tgttcaacct catttggatg gaacatatca ttacaatctt | 480 |
| ggtagggctc ttgctccatt gaagaacgat ggtgttttga ttatcggttc tggatcagct | 540 |
| actcatccac ttgatgagac acctcattat ttcgatggag ttgctccatg ggctgctgct | 600 |
| tttgattcat ggcttaggaa agctttgatc aatggtagat tcgaagaggt taacatctat | 660 |
| gaatctaagg ctcctaactg gaagcttgct catccattcc ctgagcattt ttacccactt | 720 |
| catgttgttt tgggtgctgc tggagaaaag tggaaagctg agcttattca ttcttcatgg | 780 |
| gatcatggta ccttgtgcca tggatcatac aagttcacat ctgct | 825 |

| | |
|---|---|
| <210> | 3 |
| <211> | 66 |
| <212> | DNA |
| <213> | Artificial Sequence |
| <400> | 3 |

| | |
|---|---|
| ggttcaggag ctacaaattt ttctcttttg aaacaagctg gagatgttga agagaaccca | 60 |
| ggacct | 66 |

| | |
|---|---|
| <210> | 4 |
| <211> | 50 |
| <212> | DNA |
| <213> | Artificial Sequence |
| <400> | 4 |

| | |
|---|---|
| tttacgaacg atagactagt atggatcatg ctactcttgc tatgattttg | 50 |

| | |
|---|---|
| <210> | 5 |
| <211> | 47 |
| <212> | DNA |
| <213> | Artificial Sequence |
| <400> | 5 |

| | |
|---|---|
| gatcggggaa attctctaga tcattgcaaa gaaggctcaa gtttagc | 47 |

| | |
|---|---|
| <210> | 6 |
| <211> | 39 |
| <212> | DNA |
| <213> | Artificial Sequence |
| <400> | 6 |

| | |
|---|---|
| tttacgaacg atagactagt atggtgagca agggcgagg | 39 |

| | |
|---|---|
| <210> | 7 |
| <211> | 23 |
| <212> | DNA |
| <213> | Artificial Sequence |
| <400> | 7 |

| | |
|---|---|
| cttatacagc tcgtccatgc cgt | 23 |

| | |
|---|---|
| <210> | 8 |
| <211> | 47 |
| <212> | DNA |
| <213> | Artificial Sequence |
| <400> | 8 |

| | |
|---|---|
| gcatggacga gctgtataag ggttcaggag ctacaaattt ttctctt | 47 |

| | |
|---|---|
| <210> | 9 |
| <211> | 47 |
| <212> | DNA |
| <213> | Artificial Sequence |
| <400> | 9 |

| | |
|---|---|
| cgatcgggga aattctctag attaagcaga tgtgaacttg tatgatc | 47 |

| | |
|---|---|
| <210> | 10 |
| <211> | 44 |
| <212> | DNA |
| <213> | Artificial Sequence |
| <400> | 10 |

| | |
|---|---|
| tttacgaacg atagactagt atgaagatga tgaatggtga agat | 44 |

| | |
|---|---|
| <210> | 11 |
| <211> | 47 |
| <212> | DNA |
| <213> | Artificial Sequence |
| <400> | 11 |

| | |
|---|---|
| cgatcgggga aattctctag attaagcaga tgtgaacttg tatgatc | 47 |

1. A visual plasmid for replacing a fluorescent protein, wherein the visual plasmid is a pUC-tdDODA plasmid or a pUC-DODA plasmid;
wherein genes for replacing the fluorescent protein in the pUC-tdDODA plasmid;
comprise, the tdTomato gene shown in SEQ ID NO.1 and the DODA gene shown in SEQ ID NO.2;
wherein genes for replacing the fluorescent protein in the pUC-DODA plasmid; comprise, the DODA gene shown in SEQ ID NO.2;
wherein a vector skeleton of the visual plasmid comprises a 35S promoter and a NOS terminator;
wherein the tdTomato gene and the DODA gene in the pUC-tdDODA plasmid are connected by the DNA linkage unit shown in SEQ ID NO. 3;
wherein, during construction of the plasmid pUC-tdDODA, primers used for amplifying the tdTomato gene are shown in SEQ ID NO.6 and SEQ ID NO.7;
wherein, during construction of the plasmid pUC-tdDODA, primers used for amplifying the DODA gene with the DNA linkage unit as described above are shown in SEQ ID NO.8 and SEQ ID NO.9;
wherein, primers used for amplifying the DODA gene are shown in SEQ ID NO.10 and SEQ ID NO. 11;
wherein, a process for making visual plasmid for replacing a fluorescent protein, wherein the visual plasmid is the pUC-tdDODA plasmid comprises:
using a PLV23 (TDTOMATO) plasmid as a DNA template to amplify TDTOMATO and use primer pairs as shown in SEQ. ID. NO 6 and SEQ. ID. NO. 7 in the PCR reaction:
PUC-TD F:
SEQ ID NO. 6
5′-TTTACGAACGATAGACTAGTATGGTGAGCAAGGGCGAGG-3′,;
MID-TD R:
SEQ ID NO. 7
5′-CTTATACAGCTCGTCCATGCCGT-3′,.
using a RUBY plasmid as a DNA template to amplify F2A and DODA, to use primer pairs as shown in SEQ. ID NO. 8 and SEQ. ID NO. 9 in the PCR reaction:
MID-DODA F:
SEQ ID NO. 8
5′-GCATGGACGAGCTGTATAAGGGTTTCAGGAGCTACAAATTTTTCTC
TT-3′,;
PUC-DODA R:
SEQ ID NO. 9
5′-CGATCGGGGAAATTCTCTAGATTAAGCAGATGTGAACTTGTATGA
TC-3′,;
recovering amplified PCR products by using an agarose gel DNA recovery kit;
wherein the vector has a skeleton plasmid of pUC-2×35S-EGFP, and using Restriction enzymes SpeI and XbaI to linearize the vector and cut out the EGFP gene;
wherein a process for making visual plasmid for replacing a fluorescent protein, wherein the visual plasmid is the pUC-DODA plasmid comprises:
using a Ruby plasmid as a DNA template to amplify DODA and using primer pairs as shown in SEQ. ID. NO. 10 and SEQ. ID. NO. 11 in the PCR reaction:
PUC-DODA F:
SEQ ID NO. 10
5′-TTTACGAACGATAGACTAGTATGAAGATGATGAATGGTGAAGAT-
3′,;
PUC-DODA R:
SEQ ID NO. 11
5′-CGATCGGGGAAATTCTCTAGATTAAGCAGATGTGAACTTGTATGAT
C-3′,;
recovering the two amplified PCR products by using the agarose gel DNA recovery kit;
wherein the skeleton plasmid of the vector is pUC-2×35S-EGFP, and using restriction enzymes SpeI and XbaI to linearize the vector and cut out the EGFP gene;
wherein construction of the pUC-tdDODA plasmid and the pUC-DODA plasmid further comprises:
a PCR reaction system comprises: Max Super-Fidelity DNA Polymerase 1 μL; 2×Max buffer 10 μL; dNTP Mix 0.5 μL; upstream primers PUC-TD F, MID-DODA F and PUC-DODA F RUBY F 0.8 μL; template DNA 1 μL; double distilled water 20 μL;
wherein the PCR amplification steps comprise: (1) predenaturation at 95° C. for 5 min with a cycle number of 1; (2) denaturation 95° C. for 15 sec with a cycle number of 33; (3) annealing at 58° C. for 15 sec with a cycle number of 33; (4) extension at 72° C. for 45 sec with a cycle number of 33; and (5) extension at 72° C. for 5 min with a cycle number of 1; and
wherein the enzyme digestion system comprises:
10× enzyme digestion buffer 10 μL; SpeI 1 μL; XbaI 1 μL; vector 2000 ng; double distilled water 100 μL; and
placing a reaction system in a 37° C. incubator for enzyme digestion overnight.

2-7. (canceled)


SEQUENCE LISTING (11 sequences)

SEQ ID NO: 1 - DNA, 1428 bp, synthetic construct
  source 1..1428: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtataag

SEQ ID NO: 2 - DNA, 825 bp, synthetic construct
  source 1..825: mol_type = other DNA
atgaagatgatgaatggtgaagatgctaacgatcaaatgatcaaggagtctttctttatcacccatggaaaccctatccttactgttgaagatacacatccattgaggcctttctttgaaacttggagagagaaaatcttctcaaagaaacctaaggctattcttattatctctggtcattgggagaccgttaagccaactgttaatgctgttcatatcaacgatactatccatgatttcgatgattaccctgctgctatgtatcaattcaagtacccagctcctggagaaccagagcttgctaggaaggttgaagagatcttgaagaaatcaggttttgaaacagctgagaccgatcaaaagagaggtttggatcatggagcttgggttcctttgatgcttatgtacccagaagctgatattcctgtttgtcaactttctgttcaacctcatttggatggaacatatcattacaatcttggtagggctcttgctccattgaagaacgatggtgttttgattatcggttctggatcagctactcatccacttgatgagacacctcattatttcgatggagttgctccatgggctgctgcttttgattcatggcttaggaaagctttgatcaatggtagattcgaagaggttaacatctatgaatctaaggctcctaactggaagcttgctcatccattccctgagcatttttacccacttcatgttgttttgggtgctgctggagaaaagtggaaagctgagcttattcattcttcatgggatcatggtaccttgtgccatggatcatacaagttcacatctgct

SEQ ID NO: 3 - DNA, 66 bp, synthetic construct
  source 1..66: mol_type = other DNA
ggttcaggagctacaaatttttctcttttgaaacaagctggagatgttgaagagaacccaggacct

SEQ ID NO: 4 - DNA, 50 bp, synthetic construct
  source 1..50: mol_type = other DNA
tttacgaacgatagactagtatggatcatgctactcttgctatgattttg

SEQ ID NO: 5 - DNA, 47 bp, synthetic construct
  source 1..47: mol_type = other DNA
gatcggggaaattctctagatcattgcaaagaaggctcaagtttagc

SEQ ID NO: 6 - DNA, 39 bp, synthetic construct
  source 1..39: mol_type = other DNA
tttacgaacgatagactagtatggtgagcaagggcgagg

SEQ ID NO: 7 - DNA, 23 bp, synthetic construct
  source 1..23: mol_type = other DNA
cttatacagctcgtccatgccgt

SEQ ID NO: 8 - DNA, 47 bp, synthetic construct
  source 1..47: mol_type = other DNA
gcatggacgagctgtataagggttcaggagctacaaatttttctctt

SEQ ID NO: 9 - DNA, 47 bp, synthetic construct
  source 1..47: mol_type = other DNA
cgatcggggaaattctctagattaagcagatgtgaacttgtatgatc

SEQ ID NO: 10 - DNA, 44 bp, synthetic construct
  source 1..44: mol_type = other DNA
tttacgaacgatagactagtatgaagatgatgaatggtgaagat

SEQ ID NO: 11 - DNA, 47 bp, synthetic construct; identical to SEQ ID NO: 9
  source 1..47: mol_type = other DNA